(19) United States
(12) Patent Application Publication
(10) Pub. No.: US 2025/0206750 A1
(43) Pub. Date: Jun. 26, 2025

(54) NOVEL METHOD FOR PRODUCING RUCAPARIB THAT IS PARP INHIBITOR AND INTERMEDIATE THEREOF

(71) Applicant: KOREA UNIVERSITY RESEARCH AND BUSINESS FOUNDATION, Seoul (KR)

(72) Inventors: Cheol-Hong CHEON, Seoul (KR); Jinjae PARK, Seoul (KR)

(73) Assignee: KOREA UNIVERSITY RESEARCH AND BUSINESS FOUNDATION, Seoul (KR)

(21) Appl. No.: 18/846,556

(22) PCT Filed: Jul. 28, 2022
(86) PCT No.: PCT/KR2022/011115

§ 371 Date: Sep. 12, 2024

(30) Foreign Application Priority Data
Aug. 3, 2021 (KR) 10-2021-0101907

(51) Int. Cl. C07D 487/06

(52) U.S. Cl. CPC C07D 487/06

(57) ABSTRACT

The present disclosure relates to a novel method for preparing rucaparib capable of achieving excellent synthesis yield and reproducibility. In particular, the present disclosure relates to a novel preparation method for synthesizing rucaparib by first synthesizing an indole skeleton to which substituents are introduced to positions 2, 3, 4 and 6, and then performing a heptagonal lactam ring formation reaction between the substituents introduced to positions 3 and 4, and a novel intermediate that may be used in preparation thereof.

### TECHNICAL FIELD

The present disclosure relates to a novel method for preparing rucaparib capable of achieving excellent synthesis yield and reproducibility. In particular, the present disclosure relates to a novel preparation method for synthesizing rucaparib by first synthesizing an indole skeleton to which substituents are introduced to positions 2, 3, 4 and 6, and then performing a heptagonal lactam ring formation reaction between the substituents introduced to positions 3 and 4, and a novel intermediate that may be used in preparation thereof.

### BACKGROUND ART

Rucaparib (trade name: rubraca) is an anticancer drug approved as a treatment for ovarian cancer through poly(ADP-ribose) polymerase (PARP) inhibition in late 2016 by the US FDA. After the approval by the US FDA in 2016, rucaparib was approved for use in Europe as well in 2017, and has been used as a treatment for ovarian cancer through PARP inhibition in the US and five European countries (UK, Germany, France, Italy and Spain) since 2018. In addition to ovarian cancer, rucaparib was also approved for prostate cancer in 2020, and is in extensive preclinical stages for many other types of cancer, including breast cancer.

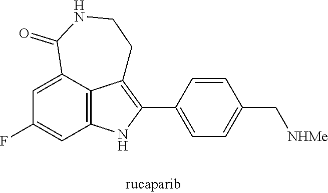 [SMILES: CNCc1ccc(-c2[nH]c3cc(F)cc4c3c2CCNC4=O)cc1]

Currently, rucaparib is mass produced through a synthetic route (Scheme 1) developed by Pfizer Inc. in 2012, however, this synthetic route proceeds through a linear sequence, and in some steps, synthesis yield is somewhat low and there is a problem with reproducibility. In particular, considering the marketability of rucaparib as a treatment for ovarian cancer and that possibility of rucaparib as a targeted anticancer drug for other types of cancer using a similar anticancer mechanism is investigated, development of a novel synthetic method for a rucaparib compound is necessary.

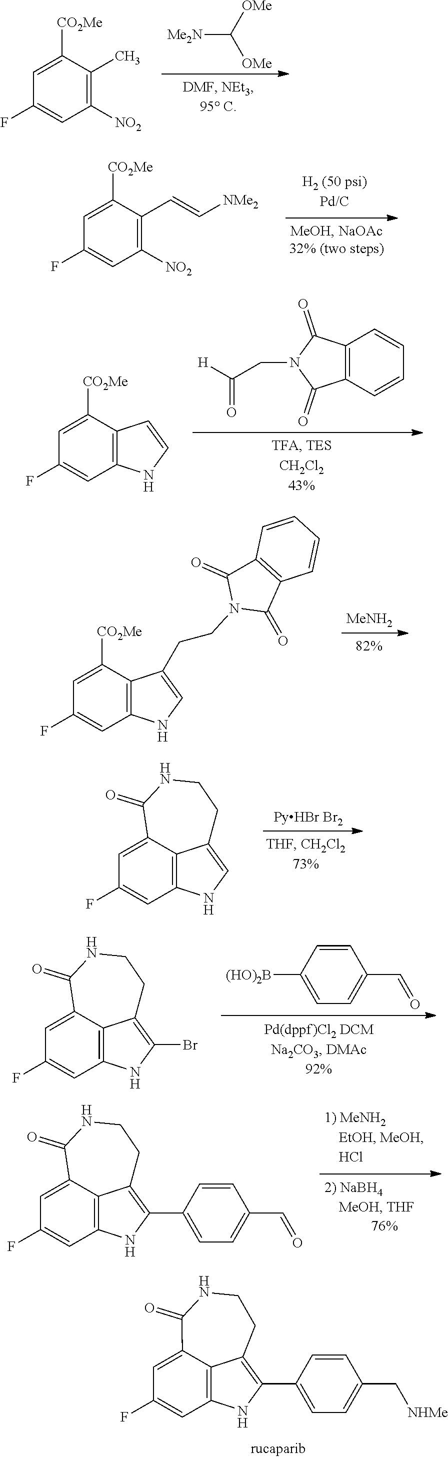

### Scheme 1. Synthetic Route Developed by Pfizer Inc.

Recognizing the need for developing such a novel synthetic method, numerus patents and papers on novel synthetic methods of this anticancer drug have been published after the FDA approval in 2016, however, most of them have focused on the synthesis of indoloazepine compound, a key intermediate in the existing synthetic method, and synthetic methods significantly improving the synthetic route have not been developed.

In view of the above, the inventors of the present disclosure have found that excellent synthesis yield and reproducibility are able to be achieved when synthesizing rucaparib by, unlike the previous synthetic route, first synthesizing an indole skeleton to which substituents are introduced to positions 2, 3, 4 and 6, and then performing a heptagonal lactam ring formation reaction between the substituents introduced to positions 3 and 4, and have completed the present disclosure.

### DISCLOSURE

### Technical Problem

The present disclosure is directed to providing a novel method for preparing rucaparib capable of achieving excellent synthesis yield and reproducibility.

The present disclosure is also directed to providing a novel intermediate that may be used in preparation of rucaparib.

### Technical Solution

In view of the above, an embodiment of the present disclosure provides a method for preparing a compound of Chemical Formula (3), the method including: reacting a compound of the following Chemical Formula (1) and a compound of the following Chemical Formula (2); and converting the result into a compound of the following Chemical Formula (3) in the presence of a catalyst:

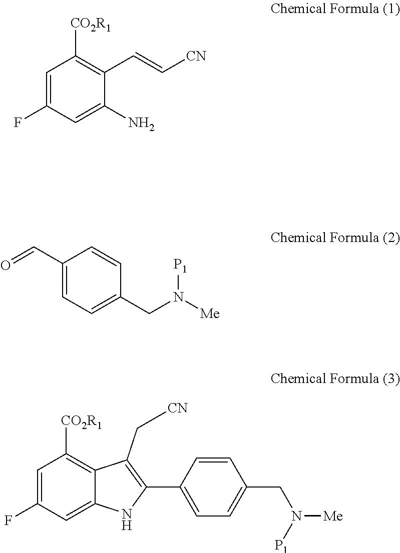

herein,
R₁ is a linear or branched C₁-C₅ alkyl; and
P₁ is, as an amine protecting group, selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

The term “C₁-C₅ alkyl” used in the present disclosure means a hydrocarbon having 1 to 5 carbon atoms, and the term “linear or branched” means that the hydrocarbon includes normal, secondary or tertiary carbon atoms. Specifically, proper examples of the “C₁-C₅ alkyl” include methyl, ethyl, 1-propyl (n-propyl), 2-propyl, 1-butyl, 2-methyl-1-propyl, 3-pentyl and the like, but are not limited thereto.

The term “protecting group” used in the present disclosure refers to a moiety of a compound completely shielding or altering properties of a functional group or properties of the compound. Chemical substructures of a protecting group are very diverse. One function of a protecting group is to act as an intermediate in the synthesis of a parent drug substance. Chemical protecting groups and strategies for protection/deprotection are widely known in the related art. Regarding this, literatures [“Protective Groups in Organic Chemistry”, Theodora W. Greene (John Wiley & Sons, Inc., New York, 1991)] and [Protective Groups in Organic Chemistry, Peter G. M. Wuts and Theodora W. Greene, 4th Ed., 2006] are referenced. Protecting groups are often used to shield reactivity of a particular functional group and assist in efficiency of a target chemical reaction. Protection of a functional group of a compound alters other physical properties other than reactivity of the protected functional group, such as polarity, hydrophobicity, hydrophilicity, and other properties that may be measured using common analytical tools. Chemically protected intermediates may be biologically and chemically active or inactive as they are. The term “amine protecting group” refers to a protecting group useful for protecting an amine group (—NH₂).

In a preferred embodiment of the present disclosure, preferred examples of the “amine protecting group” include methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc), however, the amine protecting group is not limited thereto, and protecting groups that may perform a role chemically equivalent to the protecting group are included in the category of the present disclosure.

In an embodiment of the present disclosure, the step of conversion into the compound of Chemical Formula (3) is preferably performed in the presence of a dehydrating agent. Using such a dehydrating agent may facilitate the overall reaction by removing water molecules generated when forming an imine intermediate.

In an embodiment of the present disclosure, preferred examples of the dehydrating agent include at least one compound selected from the group consisting of TiCl₄, MgSO₄ and Na₂SO₄, but are not limited thereto. In addition, in an embodiment of the present disclosure, the step of conversion into the compound of Chemical Formula (3) may be performed by reacting with molecular sieves, or using an azeotropic distillation method.

In a preferred embodiment of the present disclosure, the catalyst used in the step of conversion into the compound of Chemical Formula (3) is MCN or N-heterocyclic carbene, and herein, M is an alkali metal or NR₄⁺, and R is H or a linear or branched C₁-C₅ alkyl. The catalyst performs a role of facilitating a reaction in which an imine intermediate produced in the middle of the reaction forms an indole skeleton through cyclization.

In a preferred embodiment of the present disclosure, preferred examples of the N-heterocyclic carbene include a compound selected from the group consisting of imidazolium, triazolium and thiazolium, but are not limited thereto.

In addition, an embodiment of the present disclosure provides a method for preparing rucaparib, a compound of the following Chemical Formula (5), the method including:
(a) reacting a compound of the following Chemical Formula (1) and a compound of the following Chemical Formula (2), and converting the result into a compound of the following Chemical Formula (3) in the presence of a catalyst;
(b) performing a reaction of reducing the compound of Chemical Formula (3) to convert the result into a compound of the following Chemical Formula (4); and
(c) subjecting the compound of Chemical Formula (4) to a lactam ring formation reaction, and to deprotection before, after or simultaneously with the reaction to obtain a compound of the following Chemical Formula (5):

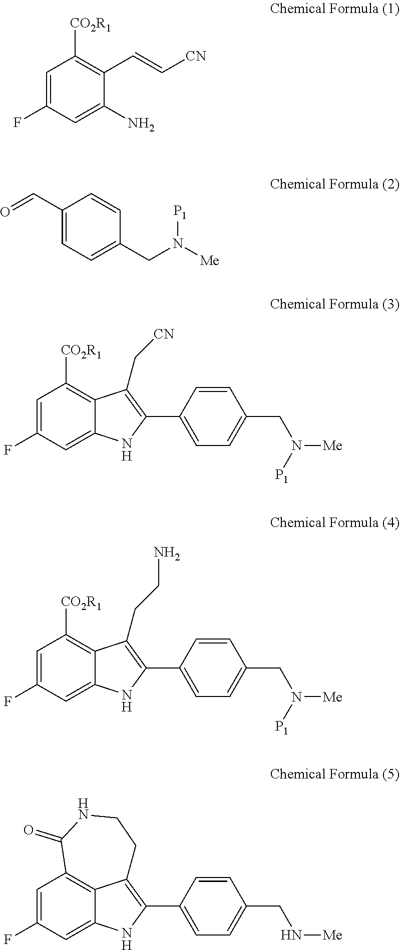

herein,
R₁ is a linear or branched C₁-C₅ alkyl; and
P₁ is, as an amine protecting group, selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

In an embodiment of the present disclosure, the reduction reaction of the step (b) is preferably performed in the presence of nickel boride.

In an embodiment of the present disclosure, the reduction reaction of the step (b) is preferably a hydrogenation reaction performed in the presence of a metal catalyst selected from the group consisting of Ni, Pd and Pt.

In an embodiment of the present disclosure, the reduction reaction of the step (b) is preferably performed in the presence of a metal catalyst selected from the group consisting of Ni, Zn, Fe and Co, and a silane compound, or performed in the presence of a metal hydride selected from the group consisting of DIBAL-H, L-selectride, NaBH₄ and borane.

In a preferred embodiment of the present disclosure, examples of the silane compound used in the present disclosure include PhSiH₃, Ph₂SiH₂, Ph₃SiH, (EtO)₃SiH, Et₃SiH, Me₂SiHSiHMe₂, PMHS (polymethylhydrosiloxane), TMDS (1,1,3,3-tetramethyldisiloxane) and the like, but are not limited thereto.

In an embodiment of the present disclosure, the deprotection of the step (c) is performed under an acidic condition, and this may be performed before, after or simultaneously with the lactam ring formation reaction to obtain rucaparib.

In an embodiment of the present disclosure, the lactam ring formation reaction of the step (c) may be performed at any suitable temperature. For example, the reaction is preferably performed at −78° C. to 100° C., −50° C. to 150° C., −25° C. to 100° C., 0° C. to 100° C., room temperature to 100° C., 50° C. to 100° C. or 50° C. to 150° C.

In a preferred embodiment of the present disclosure, the method for preparing rucaparib optionally further includes, after the step (b), (b1) protecting the compound of Chemical Formula (4) with an amine protecting group (P₂) to convert the result into a compound of Chemical Formula (9):

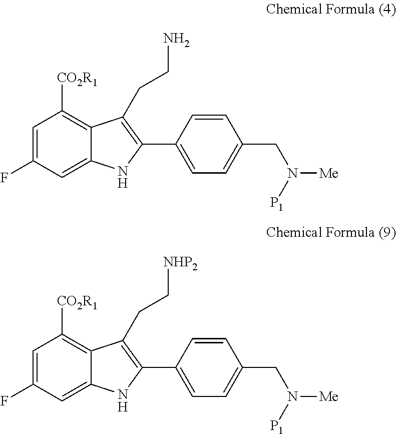

herein,
R₁ is a linear or branched C₁-C₅ alkyl; and
P₁ and P₂ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

In an embodiment of the present disclosure, the method for preparing rucaparib further includes (b2) performing a reaction of deprotecting the compound of Chemical Formula (9) after the step (b1), and includes performing the lactam ring formation reaction of the step (c) after or simultaneously with the step (b2) to obtain the compound of Chemical Formula (5):

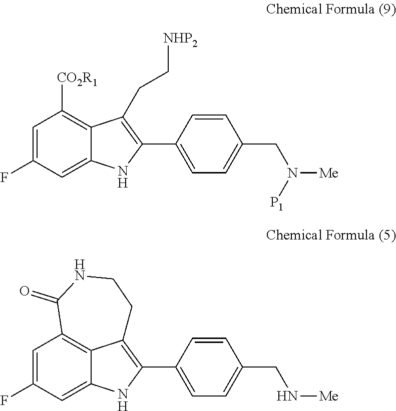

herein,
R₁ is a linear or branched C₁-C₅ alkyl; and
P₁ and P₂ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

An embodiment of the present disclosure provides a method for preparing a compound of Chemical Formula (8), the method including reacting a compound of the following Chemical Formula (6) and a compound of the following Chemical Formula (7) in the presence of a catalyst:

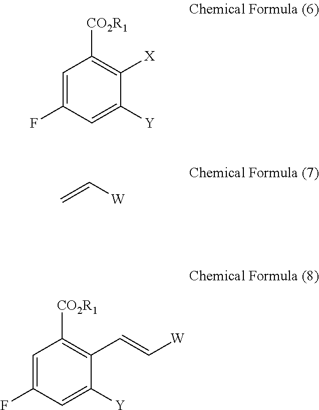

herein,
R₁ is a linear or branched C₁-C₅ alkyl;
Y is NO₂ or NH₂;
X is selected from the group consisting of OTf, OTs, OMs, I, Br and Cl;
W is selected from the group consisting of —CN, —COOR₂, —CONH₂, —CONHP₂ and —CONP₂P₃;
R₂ is H or a linear or branched C₁-C₅ alkyl; and
P₁, P₂ and P₃ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

In a preferred embodiment of the present disclosure, the catalyst used in the method for preparing the compound of Chemical Formula (8) is preferably a metal catalyst, and more preferably Ni or Pd.

In addition, in a preferred embodiment of the present disclosure, the metal catalyst is preferably a common Pd catalyst, and more preferred examples thereof include catalysts selected from among Pd(PtBu₃)₂, Pd(PPh₃)₄, Pd(PPh₃)₂Cl₂ and Pd(OAc)₂+ligand combinations (herein, the ligand is Buchwald ligand such as SPhos, XPhos, JohnPhos and DavePhos), but are not limited thereto.

In a preferred embodiment of the present disclosure, the method for preparing the compound of Chemical Formula (8) further includes, when Y is NO₂, converting the NO₂ into NH₂ through a reduction reaction. For example, when Y is NO₂ in the compound of Chemical Formula (6) and W is —CN, NO₂ may be reduced to NH₂ through a reduction reaction to obtain the compound of Chemical Formula (1).

In addition, an embodiment of the present disclosure provides, as a novel intermediate that may be used in preparing rucaparib of the present disclosure, a compound of the following Chemical Formula (1) or a compound of the following Chemical Formula (3):

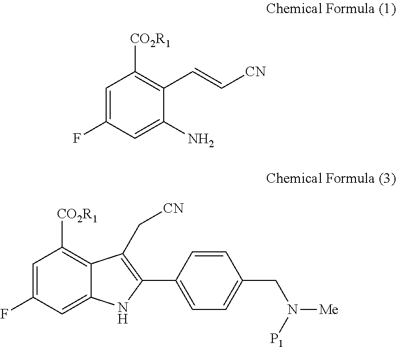

herein,
R₁ is a linear or branched C₁-C₅ alkyl; and
P₁ is, as an amine protecting group, selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

Any suitable solvent may be used in the method of the present disclosure. Representative solvents include pentane, pentanes, hexane, hexanes, heptane, heptanes, petroleum ether, cyclopentane, cyclohexane, benzene, toluene, xylene, dichloromethane, trifluoromethylbenzene, halobenzene such as chlorobenzene, fluorobenzene, dichlorobenzene and difluorobenzene, methylene chloride, chloroform, acetone, ethyl acetate, diethyl ether, tetrahydrofuran (THF), 2-methyltetrahydrofuran, dibutyl ether, diisopropyl ether, methyl tert-butyl ether, dimethoxyethane, dioxane (1,4 dioxane), N-methyl pyrrolidinone (M4P), DMF, alcohol such as methanol, ethanol, propanol and butanol, or mixtures thereof, but are not limited thereto.

The reaction mixture of each step of the present disclosure may be under any suitable pressure. For example, the reaction mixture may be under atmospheric pressure. The reaction mixture may also be exposed to any suitable environment such as atmospheric gas, or inert gas such as nitrogen or argon.

The reaction of each step of the present disclosure may be performed at any suitable temperature. For example, the temperature of the mixture during the reaction may be from −78° C. to 100° C., −50° C. to 150° C., −25° C. to 100° C., 0° C. to 100° C., room temperature to 100° C. or 50° C. to 100° C.

### Advantageous Effects

According to the present disclosure, it is possible to obtain a novel preparation method for synthesizing rucaparib by first synthesizing an indole skeleton to which substituents are introduced to positions 2, 3, 4 and 6, and then performing a heptagonal lactam ring formation reaction between the substituents introduced to positions 3 and 4, and a novel intermediate that may be used in preparation thereof.

### BRIEF DESCRIPTION OF DRAWINGS

FIG. 1 is an NMR spectrum of (E)-2-amino-4-fluoro-6-methoxycarbonylcinnamyl nitrile (Compound 3).

FIG. 2 is an NMR spectrum of 2-(4-((N-tert-butoxycarbonyl)-(N-methyl)aminomethyl)phenyl)-6-fluoro-4-methoxycarbonyl-indole-3-acetonitrile (Compound 2).

FIG. 3 is an NMR spectrum of rucaparib (Compound 1).

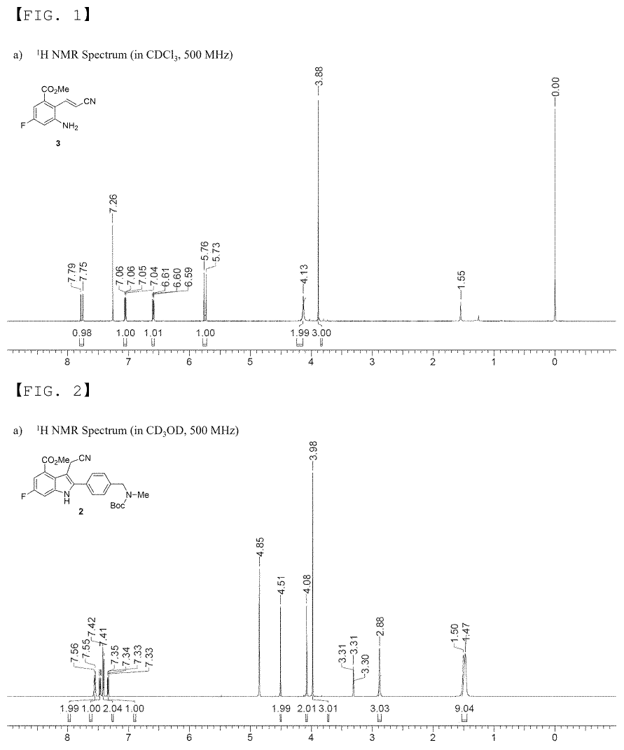

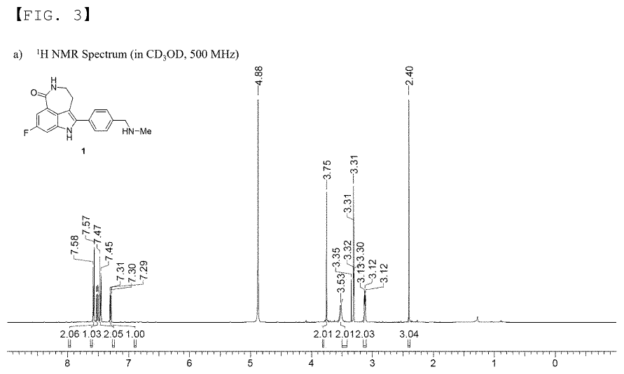

### BEST MODE

Hereinafter, the present disclosure will be described in more detail with reference to examples. However, the following examples are provided to help understand the present disclosure, and the scope of the present disclosure is not limited to the following examples.

### General Procedure

Unless otherwise stated, all reactions were performed in oven-dried glassware under an argon atmosphere. Unless otherwise indicated, all reactions were magnetically stirred, monitored by analytical thin layer chromatography (TLC) using a silica gel glass plate (0.25 mm) pre-coated with an F254 indicator, and visualized with UV light (254 nm). Flash column chromatography was performed using silica gel 60 (230 mesh to 400 mesh) with an indicated eluent. Commercial grade reagents were used without further purification. Unless otherwise stated, a yield refers to chromatographically and spectroscopically pure compound. 1H NMR and ¹³C NMR spectra were recorded on 500 MHz and 125 MHz spectrometers, respectively. Tetramethylsilane (δ_{TMS}: 0.0 ppm) and residual NMR solvent (CDCl₃ (δ_{H}: 7.26 ppm, δ_{C}: 77.16 ppm) or (CD₃)₂SO (δ_{H}: 2.50 ppm, δ_{C}: 39.52 ppm) were used as internal standards for 1H NMR and ¹³C NMR spectra, respectively. A proton spectrum was expressed as δ (proton position, multiplicity, coupling constant J, number of protons). Multiplicity was expressed as s (singlet), d (doublet), t (triplet), q (quartet), p (quintet), m (multiplet) and br (broad). A high-resolution mass spectrum (HRMS) was recorded on a quadrupole time-of-flight mass spectrometer (QTOF-MS) using electrospray ionization (ESI) as an ionization method.

Main reagents used are TiCl₄, NaBH₄, methanol (MeOH: for analysis, ACS grade, Carlo Erba Reagents) and the like.

### Synthesis Example 1: Method for Preparing Intermediate Through Heck Coupling Reaction

### Synthesis Example 1-1: (E)-2-amino-4-fluoro-6-methoxycarbonylcinnamyl nitrile (Compound 3)

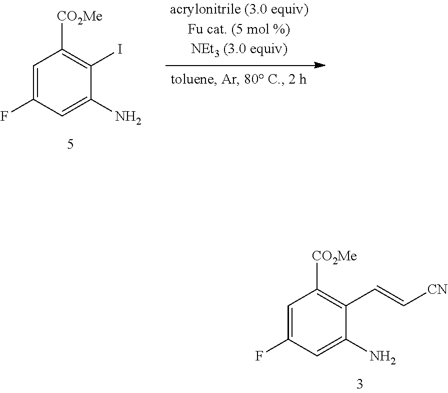 [SMILES: COC(=O)c1cc(F)cc(N)c1I ; COC(=O)c1cc(F)cc(N)c1/C=C/C#N]

Acrylonitrile (2.0 mL, 30 mmol) was added to a toluene (100 mL) solution of Compound 5 (2.95 g, 10 mmol), Fu catalyst (Pd(PtBu₃)₂; 255 mg, 0.5 mmol) and triethylamine (4.2 mL, 30 mmol), and while stirring the reaction mixture at 80° C., the progress of the reaction was observed by TLC. After Compound 5 was completely consumed, the reaction mixture was filtered through Celite to remove insoluble solids, and then concentrated. To the mixture, a saturated aqueous NH₄Cl solution (100 mL) was added dropwise, and then the obtained mixture was extracted three times using ethyl acetate (100 mL). The obtained organic layer was dried with MgSO₄, and then concentrated. The reaction mixture was dissolved again in ethyl acetate, and then hexane was added dropwise thereto to produce white precipitates. The obtained precipitates were filtered to obtain Compound 3 (1.9 g, 8.8 mmol, 88%), a yellow solid.

¹H NMR (500 MHz, CDCl₃) δ 7.77 (d, J=17.1 Hz, 1H), 7.05 (dd, J=8.8, 2.5 Hz, 1H), 6.60 (dd, J=9.7, 2.5 Hz, 1H), 5.74 (d, J=16.9 Hz, 1H), 4.13 (br, 2H), 3.88 (s, 3H); ¹³C{¹H}NMR (125 MHz, CDCl₃) δ 166.4 (d, J=3.6 Hz), 163.1 (d, J=247.9 Hz), 148.1, 146.9 (d, J=10.9 Hz), 132.5 (d, J=9.1 Hz), 117.7, 116.1 (d, J=3.6 Hz), 108.1 (d, J=24.5 Hz), 106.4 (d, J=24.5 Hz), 101.7, 52.8; ¹⁹F NMR (471 MHz, CDCl₃) δ −110.0; HRMS (ESI-TOF) m/z: [M+H]⁺ calcd for C₁₁H₁₀FN₂O₂ 221.0726; found 221.0728.

### Synthesis Example 1-2: (E)-N-(4-methoxybenzyl)-2-amino-4-fluoro-6-methoxycarbonylcinnamide (Compound 10)

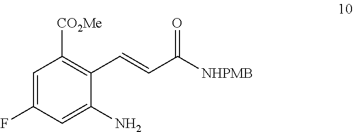

Compound 10 (80%), a white solid, was obtained through the process of Synthesis Example 1-1 using N-(4-methoxybenzyl)acrylamide instead of acrylonitrile.

¹H NMR (500 MHz, DMSO-d₆) δ 8.50 (t, J=5.9 Hz, 1H), 7.49 (d, J=16.0 Hz, 1H), 7.22 (d, J=8.7 Hz, 2H), 6.89 (d, J=8.7 Hz, 2H), 6.65 (dd, J=11.2, 2.7 Hz, 1H), 6.62 (dd, J=8.9, 2.6 Hz, 1H), 6.20 (d, J=16.0 Hz, 1H), 5.73 (s, 2H), 4.30 (d, J=6.0 Hz, 2H), 3.75 (s, 3H), 3.73 (s, 3H); ¹³C{1H}NMR (125 MHz, DMSO-d₆) δ 167.6 (d, J=3.6 Hz), 164.7, 161.9 (d, J=242.5 Hz), 158.3, 149.4 (d, J=11.8 Hz), 133.9, 133.7 (d, J=10 Hz), 131.3, 128.8, 125.8, 115.3 (d, J=2.7 Hz), 113.7, 103.4 (d, J=24.5 Hz), 103.3 (d, J=24.5 Hz), 55.1, 52.3, 41.8; ¹⁹F NMR (471 MHz, DMSO-d₆) δ −113.1; HRMS (ESI-TOF): m/z: [M+Na]⁺ calcd for C₁₉H₁₉FN₂NaO₄ 381.1221; found 381.1226.

### Synthesis Example 1-3: (E)-ethyl 2-amino-4-fluoro-6-methoxycarbonylcinnamate (Compound 9)

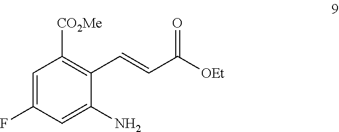 [SMILES: CCOC(=O)/C=C/c1c(N)cc(F)cc1C(=O)OC]

Compound 9 (90%), a yellow solid, was obtained through the process of Synthesis Example 1-1 using ethyl acrylate instead of acrylonitrile.

¹H NMR (500 MHz, CDCl₃) δ 7.95 (d, J=16.5 Hz, 1H), 7.00 (dd, J=8.9, 2.6 Hz, 1H), 6.57 (dd, J=9.9, 2.6 Hz, 1H), 6.18 (d, J=16.5 Hz, 1H), 4.27 (q, J=7.2 Hz, 2H), 4.18 (br, 2H), 3.86 (s, 3H), 1.33 (t, J=7.1 Hz, 3H); ¹³C{1H}NMR (125 MHz, CDCl₃) δ 166.8 (d, J=3.6 Hz), 166.6, 162.7 (d, J=247.0 Hz), 147.0 (d, J=10.9 Hz), 141.5, 132.8 (d, J=9.1 Hz), 122.7, 116.9 (d, J=2.7 Hz), 107.3 (d, J=24.5 Hz), 105.8 (d, J=24.5 Hz), 60.7, 52.5, 14.3.

### Synthesis Example 2: (E)-N-(4-methoxybenzyl)-2-amino-4-fluoro-6-methoxycarbonylcinnamide (Compound 10)

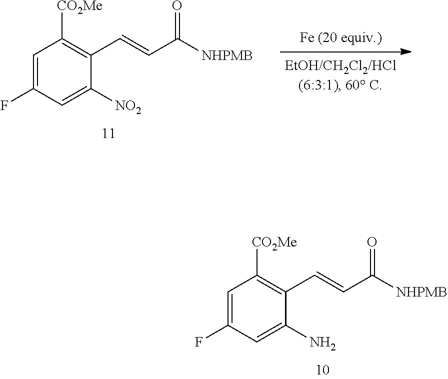

After dissolving Compound 11 (4.3 g, 11 mmol) in a mixture solution of ethanol, dichloromethane and a 35% aqueous hydrochloric acid solution (6:3:1, 180 mL), iron powder (12 g, 220 mmol) was added thereto, and while stirring the reaction mixture at 60° C., the progress of the reaction was observed by TLC. After Compound 8 was completely consumed, the reaction mixture was filtered through Celite to remove insoluble solids, and then concentrated. To the mixture, a saturated aqueous NH₄Cl solution (200 mL) was added dropwise, and then the obtained mixture was extracted three times using ethyl acetate (200 mL). The obtained organic layer was dried with MgSO₄, and then concentrated. The reaction mixture was dissolved again in ethyl acetate, and then hexane was added dropwise thereto to produce white precipitates. The obtained precipitates were filtered to obtain Compound 10 (3.6 g, 10 mmol, 92%), a white solid.

¹H NMR (500 MHz, DMSO-d₆) δ 8.50 (t, J=5.9 Hz, 1H), 7.49 (d, J=16.0 Hz, 1H), 7.22 (d, J=8.7 Hz, 2H), 6.89 (d, J=8.7 Hz, 2H), 6.65 (dd, J=11.2, 2.7 Hz, 1H), 6.62 (dd, J=8.9, 2.6 Hz, 1H), 6.20 (d, J=16.0 Hz, 1H), 5.73 (s, 2H), 4.30 (d, J=6.0 Hz, 2H), 3.75 (s, 3H), 3.73 (s, 3H); ¹³C{1H}NMR (125 MHz, DMSO-d₆) δ 167.6 (d, J=3.6 Hz), 164.7, 161.9 (d, J=242.5 Hz), 158.3, 149.4 (d, J=11.8 Hz), 133.9, 133.7 (d, J=10 Hz), 131.3, 128.8, 125.8, 115.3 (d, J=2.7 Hz), 113.7, 103.4 (d, J=24.5 Hz), 103.3 (d, J=24.5 Hz), 55.1, 52.3, 41.8; ¹⁹F NMR (471 MHz, DMSO-d₆) 5-113.1; HRMS (ESI-TOF): m/z: [M+Na]⁺ calcd for C₁₉H₁₉FN₂NaO₄ 381.1221; found 381.1226.

### Synthesis Example 3: Preparation of 2-(4-((N-tert-butoxycarbonyl)-(N-methyl)aminomethyl)phenyl)-6-fluoro-4-methoxycarbonyl-indole-3-acetonitrile (Compound 2)

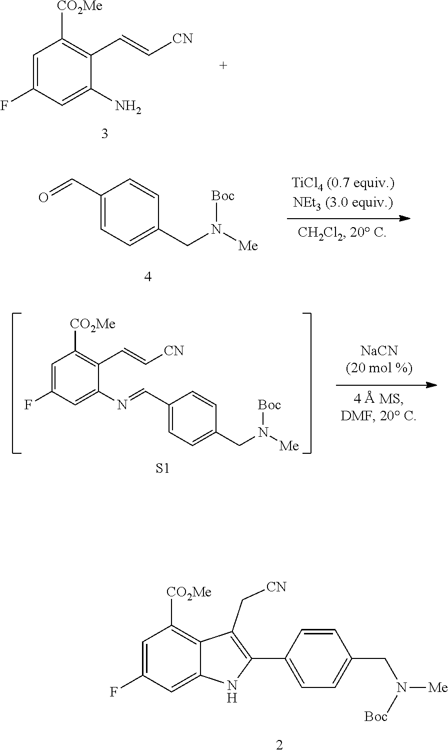

Titanium tetrachloride (1.0 M dichloromethane solution, 7.0 mL, 7.0 mmol) was added to a dichloromethane (100 mL) solution of 2-aminocinnamyl nitrile (Compound 3) (2.2 g, 10 mmol), aldehyde Compound 4 (2.5 g, 10 mmol) and triethylamine (4.2 mL, 30 mmol), and while stirring the reaction mixture at 20° C., the progress of the reaction was observed by TLC and 1H NMR analysis. After Compound 3 and Compound 4 were completely consumed, distilled water (100 mL) was added dropwise to the reaction mixture, and the obtained mixture was extracted three times using dichloromethane (100 mL). The obtained organic layer was dried with MgSO₄, and then concentrated to obtain a mixture of imine Compound S1, and it was directly used in the next reaction without further separation.

After dissolving the mixture of imine Compound S1 in dimethylformamide (100 mL), 4 Å molecular sieves (4.0 g) and sodium cyanide (98 mg, 2.0 mmol) were added to this mixture solution, and while stirring the reaction mixture at 20° C., the progress of the reaction was observed by TLC. After Compound S3 was completely consumed, insoluble solids were filtered and removed, and the filtrate was washed with ethyl acetate. The obtained filtrate was concentrated, and then the reaction mixture was separated and purified by silica-based column chromatography, in which a mixture solution of ethyl acetate and hexane (1:2) was employed as a developing solution, to obtain Compound 2 (3.6 g, 8.0 mmol, two-step yield 80%), a white solid.

¹H NMR (500 MHz, CD₃OD) δ 7.56 (d, J=7.9 Hz, 2H), 7.47 (dd, J=10.2, 2.4 Hz, 1H), 7.42 (d, J=8.1 Hz, 2H), 7.34 (dd, J=8.7, 2.4 Hz, 1H), 4.51 (s, 2H), 4.08 (s, 2H), 3.98 (s, 3H), 2.88 (br, 3H), 1.49 (d, J=15.9 Hz, 9H); ¹³C{1H}NMR (125 MHz, CD₃OD) δ 168.7, 159.6 (d, J=237.1 Hz), 157.7 (d, J=49.1 Hz), 141.5 (d, J=3.6 Hz), 140.3 (d, J=22.7 Hz), 139.1 (d, J=11.8 Hz), 131.6, 130.2, 129.0, 124.8 (d, J=9.1 Hz), 123.3, 120.9, 112.5 (d, J=26.3 Hz), 103.1 (d, J=25.4 Hz), 102.4, 81.4, 52.9 (d, J=100.8 Hz), 52.8, 34.7, 28.7, 17.0; ¹⁹F NMR (471 MHz, CD₃OD) δ −123.0; HRMS (ESI-TOF) m/z: [M+Na]⁺ calcd for C₂₅H₂₆FN₃NaO₄ 474.1805; found 474.1802.

### Synthesis Example 4: Method for Preparing Rucaparib

### Synthesis Example 4-1: Rucaparib (Compound 1)

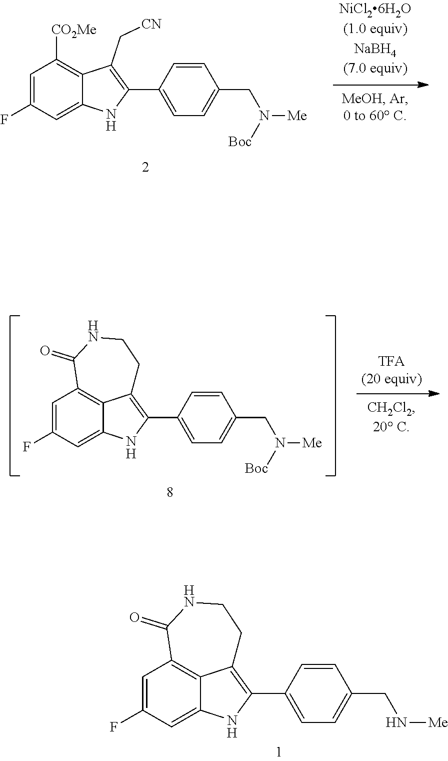

Sodium borohydride (265 mg, 7.0 mmol) was added to a methanol solution (10 mL) of Compound 2 (451 mg, 1.0 mmol) and nickel chloride hexahydrate (NiCl₂·6H₂O; 238 mg, 1.0 mmol) at 0° C., and then the reaction mixture was stirred at 60° C. After Compound 2 was completely consumed, diethylenetriamine (1.1 mL, 10 mmol) was added dropwise to the reaction mixture, and the reaction mixture was stirred for 30 minutes and then concentrated. To the mixture, a saturated aqueous NaHCO₃ solution (10 mL) was added dropwise, and then the obtained mixture was extracted three times using ethyl acetate (10 mL). The obtained organic layer was dried with MgSO₄, and then concentrated to obtain a mixture of Compound 8, and it was directly used in the next reaction without further separation.

After dissolving the mixture of Compound 8 in dichloromethane (10 mL), trifluoroacetic acid (TFA; 1.6 mL, 20 mmol) was added to this mixture solution at 20° C., and while stirring the reaction mixture, the progress of the reaction was observed by TLC. After Compound 8 was completely consumed, the reaction mixture was concentrated, and separated and purified by silica-based column chromatography, in which a mixture solution of dichloromethane, methanol and triethylamine (90:10:1) was employed as a developing solution, to obtain rucaparib 1 (236 mg, 0.73 mmol, two-step yield 73%), a white solid.

¹H NMR (500 MHz, CD₃OD) δ 7.57 (d, J=8.2 Hz, 2H), 7.51 (dd, J=10.8, 2.3 Hz, 1H), 7.46 (d, J=8.2 Hz, 2H), 7.30 (dd, J=9.0, 2.4 Hz, 1H), 3.75 (s, 2H), 3.53 (br, 2H), 3.15-3.10 (m, 2H), 2.40 (s, 3H); ¹³C{1H}NMR (125 MHz, CD₃OD) δ 172.6, 160.6 (d, J=235.2 Hz), 140.2, 138.6 (d, J=11.8 Hz), 137.3 (d, J=3.6 Hz), 132.3, 130.0, 129.2, 125.8 (d, J=9.1 Hz), 125.0, 112.9, 111.2 (d, J=26.3 Hz), 102.2 (d, J=26.3 Hz), 56.0, 43.8, 35.6, 30.0; ¹⁹F NMR (471 MHz, CD₃OD) δ −123.4; HRMS (ESI-TOF) m/z: [M+H]⁺ calcd for C₁₉H₁₉FN₃O 324.1507; found: 324.1510.

### Synthesis Example 4-2: Rucaparib (Compound 1)

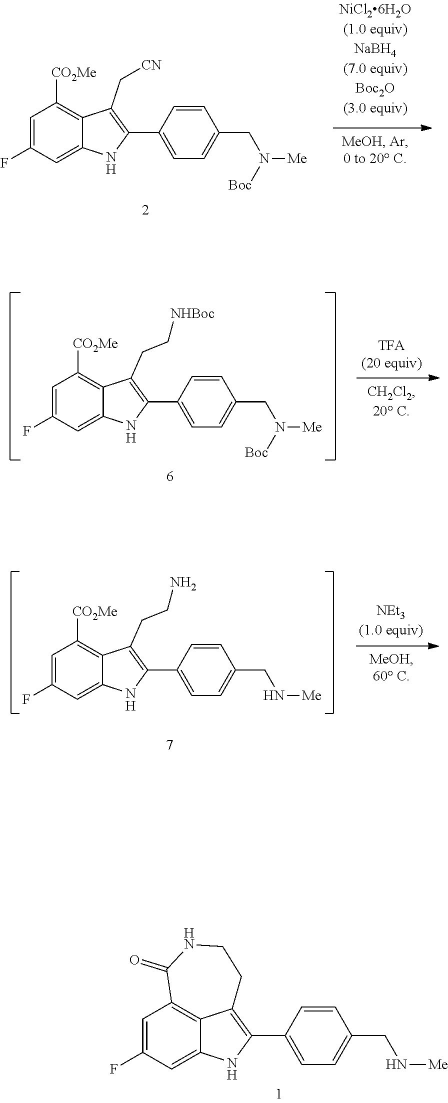

Sodium borohydride (265 mg, 7.0 mmol) was added to a methanol (10 mL) solution of Compound 2 (451 mg, 1.0 mmol), di-tert-butyl dicarbonate (Boc₂O; 655 mg, 3.0 mmol) and nickel chloride hexahydrate (NiCl₂·6H₂O; 238 mg, 1.0 mmol) at 0° C., and then the reaction mixture was stirred at 20° C. After Compound 2 was completely consumed, diethylenetriamine (1.1 mL, 10 mmol) was added dropwise to the reaction mixture, and the reaction mixture was stirred for 30 minutes and then concentrated. To the mixture, a saturated aqueous NaHCO₃ solution (10 mL) was added dropwise, and then the obtained mixture was extracted three times using ethyl acetate (10 mL). The obtained organic layer was dried with MgSO₄ and then concentrated, and some remaining by-products were removed through silica-based flash column chromatography to obtain a mixture of Compound 6, and it was directly used in the next reaction.

After dissolving the mixture of Compound 6 in dichloromethane (10 mL), trifluoroacetic acid (TFA; 1.6 mL, 20 mmol) was added to this mixture solution at 20° C., and while stirring the reaction mixture, the progress of the reaction was observed by TLC. After Compound 6 was completely consumed, the reaction mixture was concentrated to obtain a mixture of Compound 7, and it was directly used in the next reaction without further separation.

After dissolving the mixture of Compound 7 in methanol (10 mL), triethylamine (0.14 mL, 1.0 mmol) was added thereto at 20° C., and while stirring the reaction mixture at 60° C., the progress of the reaction was observed by TLC. After Compound 7 was completely consumed, the reaction mixture was concentrated, and separated and purified by silica-based column chromatography, in which a mixture solution of dichloromethane, methanol and triethylamine (90:10:1) was employed as a developing solution, to obtain rucaparib 1 (260 mg, 0.80 mmol, three-step yield 80%), a white solid.

¹H NMR (500 MHz, CD₃OD) δ 7.57 (d, J=8.2 Hz, 2H), 7.51 (dd, J=10.8, 2.3 Hz, 1H), 7.46 (d, J=8.2 Hz, 2H), 7.30 (dd, J=9.0, 2.4 Hz, 1H), 3.75 (s, 2H), 3.53 (br, 2H), 3.15-3.10 (m, 2H), 2.40 (s, 3H); ¹³C{1H}NMR (125 MHz, CD₃OD) δ 172.6, 160.6 (d, J=235.2 Hz), 140.2, 138.6 (d, J=11.8 Hz), 137.3 (d, J=3.6 Hz), 132.3, 130.0, 129.2, 125.8 (d, J=9.1 Hz), 125.0, 112.9, 111.2 (d, J=26.3 Hz), 102.2 (d, J=26.3 Hz), 56.0, 43.8, 35.6, 30.0; ¹⁹F NMR (471 MHz, CD₃OD) δ −123.4; HRMS (ESI-TOF) m/z: [M+H]⁺ calcd for C₁₉H₁₉FN₃O 324.1507; found: 324.1510.

1. A method for preparing a compound of Chemical Formula (3), the method comprising:
reacting a compound of the following Chemical Formula (1) and a compound of the following Chemical Formula (2); and
converting the result into a compound of the following Chemical Formula (3) in the presence of a catalyst: 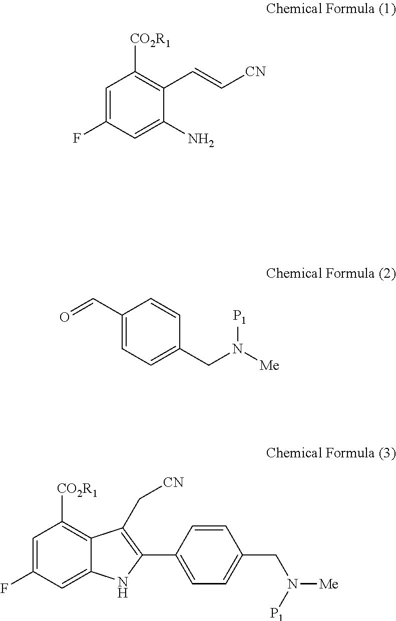
herein,
R₁ is a linear or branched C₁-C₅ alkyl; and
P₁ is, as an amine protecting group, selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

2. The method of claim 1, wherein the conversion into the compound of Chemical Formula (3) is performed in the presence of a dehydrating agent.

3. The method of claim 2, wherein the dehydrating agent is at least one compound selected from the group consisting of TiCl₄, MgSO₄ and Na₂SO₄, or a molecular sieve.

4. The method of claim 1, wherein the conversion into the compound of Chemical Formula (3) is performed using an azeotropic distillation method.

5. The method of claim 1, wherein the catalyst is MCN or N-heterocyclic carbene:
herein,
M is an alkali metal or NR₄⁺; and
R is H or a linear or branched C₁-C₅ alkyl.

6. The method of claim 5, wherein the N-heterocyclic carbene is selected from the group consisting of imidazolium, triazolium and thiazolium.

7. A method for preparing rucaparib, a compound of the following Chemical Formula (5), the method comprising:
(a) reacting a compound of the following Chemical Formula (1) and a compound of the following Chemical Formula (2), and converting the result into a compound of the following Chemical Formula (3) in the presence of a catalyst;
(b) performing a reaction of reducing the compound of Chemical Formula (3) to convert the result into a compound of the following Chemical Formula (4); and
(c) subjecting the compound of Chemical Formula (4) to a lactam ring formation reaction, and to deprotection before, after or simultaneously with the reaction to obtain a compound of the following Chemical Formula (5): 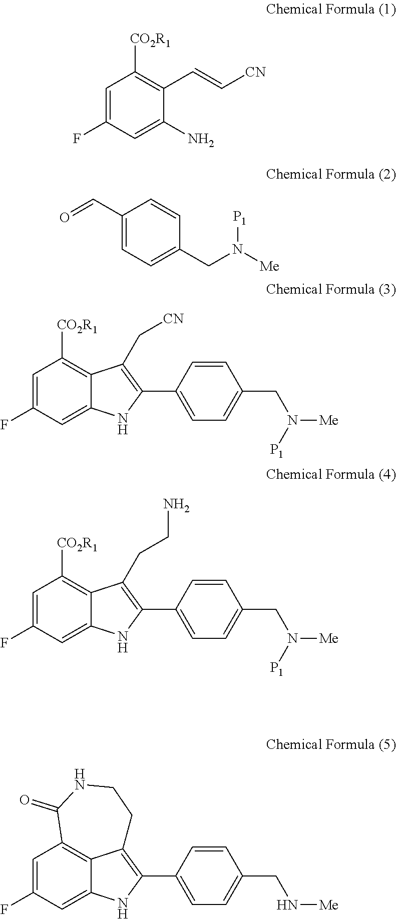
herein,
R₁ is a linear or branched C₁-C₅ alkyl; and
P₁ is, as an amine protecting group, selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

8. The method of claim 7, wherein (a) is performed in the presence of a dehydrating agent.

9. The method of claim 8, wherein the dehydrating agent is at least one compound selected from the group consisting of TiCl₄, MgSO₄ and Na₂SO₄, or a molecular sieve.

10. The method of claim 7, wherein (a) is performed using an azeotropic distillation method.

11. The method of claim 7, wherein the catalyst used in (a) is MCN or N-heterocyclic carbene:
herein,
M is an alkali metal or NR₄⁺; and
R is H or a linear or branched C₁-C₅ alkyl.

12. The method of claim 7, wherein the reduction reaction of (b) is performed in the presence of nickel boride.

13. The method of claim 7, wherein the reduction reaction of (b) is a hydrogenation reaction performed in the presence of a metal catalyst selected from the group consisting of Ni, Pd and Pt.

14. The method of claim 7, wherein the reduction reaction of (b) is performed in the presence of a metal catalyst selected from the group consisting of Ni, Zn, Fe and Co, and a silane compound.

15. The method of claim 7, wherein the reduction reaction of (b) is performed in the presence of a metal hydride selected from the group consisting of DIBAL-H, L-selectride, NaBH₄ and borane.

16. The method of claim 7, wherein the deprotection of (c) is performed under an acidic condition.

17. The method of claim 7, further comprising, (b1) protecting the compound of Chemical Formula (4) with an amine protecting group (P₂) to convert the result into a compound of Chemical Formula (9) after (b): 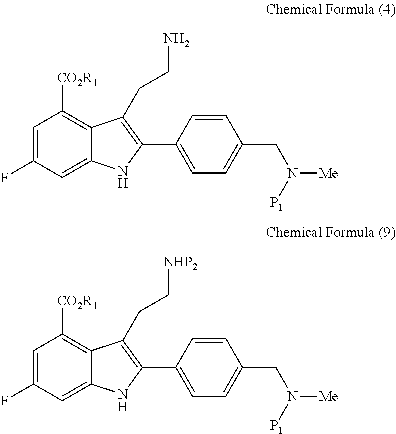
herein,
R₁ is a linear or branched C₁-C₅ alkyl; and
P₁ and P₂ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

18. The method of claim 17, further comprising (b2) performing a reaction of deprotecting the compound of Chemical Formula (9) after (b1), and performing the lactam ring formation reaction of (c) after or simultaneously with (b2) to obtain the compound of Chemical Formula (5): 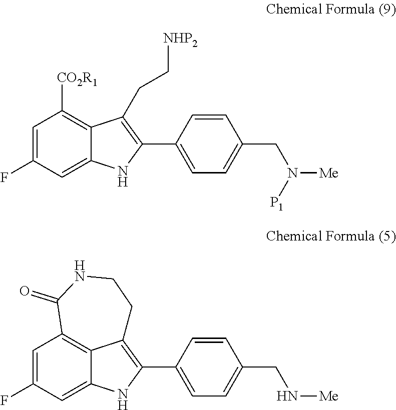
herein,
R₁ is a linear or branched C₁-C₅ alkyl; and
P₁ and P₂ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

19. A method for preparing Chemical Formula (8), the method comprising reacting a compound of the following Chemical Formula (6) and a compound of the following Chemical Formula (7) in the presence of a catalyst: 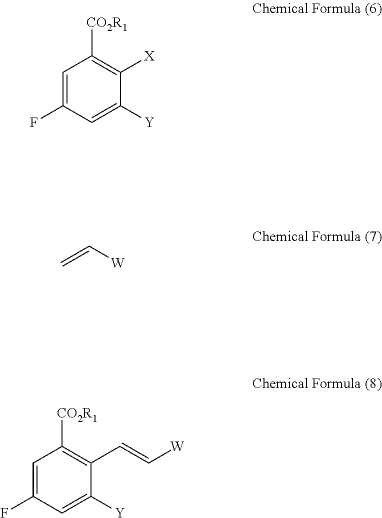
herein,
R₁ is a linear or branched C₁-C₅ alkyl;
Y is NO₂ or NH₂;
X is selected from the group consisting of OTf, OTs, OMs, I, Br and Cl;
W is selected from the group consisting of —CN, —COOR₂, —CONH₂, —CONHP₂ and —CONP₂P₃;
R₂ is H or a linear or branched C₁-C₅ alkyl; and
P₁, P₂ and P₃ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

20. The method of claim 19, further comprising, when Y is NO₂, converting the NO₂ into NH₂ through a reduction reaction.

21. The method of claim 19, wherein the catalyst is a metal catalyst selected from the group consisting of Ni and Pd.

22. A compound of the following Chemical Formula (1) used for preparing rucaparib: 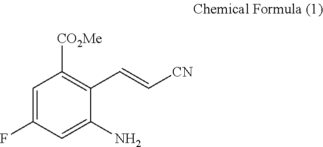 [SMILES: COC(=O)c1cc(F)cc(N)c1/C=C/C#N]
herein,
R₁ is a linear or branched C₁-C₅ alkyl.

23. A compound of the following Chemical Formula (3) used for preparing rucaparib: 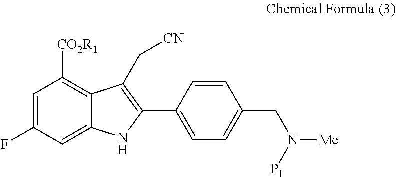
herein,
R₁ is a linear or branched C₁-C₅ alkyl; and
P₁ is, as an amine protecting group, selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).
